(19) United States
(12) Patent Application Publication
(10) Pub. No.: US 2010/0048885 A1
(43) Pub. Date: Feb. 25, 2010

(54) Immunostimulating Polysaccharides Isolated From Curcuma Xanthorrhiza and Manufacturing Method Thereof

(76) Inventors: Jae-Kwan Hwang, Gyeonggi-do (KR); Ah-Jin Kim, Seoul (KR); Jong-Hee Sohn, Seoul (KR); Kyu-Lee Han, Seoul (KR); Sun-Hee Lee, Gyeonggi-do (KR); Jeong-Han Choo, Gyeonggi-do (KR)

Correspondence Address: BUCHANAN, INGERSOLL & ROONEY PC, POST OFFICE BOX 1404, ALEXANDRIA, VA, 22313-1404, US

(21) Appl. No.: 11/922,107

(22) PCT Filed: Jun. 14, 2006
(86) PCT No.: PCT/KR2006/002282

§ 371 Date: Feb. 20, 2008

(30) Foreign Application Priority Data
Jun. 14, 2005 (KR) 10-2005-0051176

(51) Int. Cl. C07H 1/00; C07H 3/00

(52) U.S. Cl. 536/123.1

(57) ABSTRACT

The present invention provides a method for manufacturing polysaccharides isolated from Curcuma xanthorrhiza, including steps of: (S1) preparing a powder of Curcuma xanthorrhiza Roxb.; (S2) extracting the powder with an organic solvent, and then filtering or centrifuging to obtain a residue; (S3) extracting the residue to prepare a solution containing polysaccharides; (S4) removing starch by adding starch-hydrolyzing enzyme to the polysaccharides-containing solution; (S5) precipitating the polysaccharides after the step (S4); and (S6) purifying the polysaccharides after the step (S5), polysaccharides obtained according to the manufacturing method, and a pharmaceutical composition in eluding the polysaccharides as effective component. The polysaccharides according to the present invention may be very effectively used in drugs and functional foods for stimulating macrophage activity, preventing and treating immunological diseases including cancer, and enhancing immunity after the treatment of the immunological diseases.

### TECHNICAL FIELD

The present invention relates to effective polysaccharides isolated from Curcuma xanthorrhiza, a manufacturing method thereof and a use of the isolated polysaccharides.

### BACKGROUND ART

Polysaccharides having function of macrophage activation have an ability to activate macrophage, which plays an important role in biological defense mechanism cause d when body is infected with bacteria, fungi and viruses. At this time, the macrophage is classified into cellular immunity and constitutes a fundamental immune system since the macrophage reacts to immune cells such as complements, NK cell and the like with the activation of the macrophage (Plafair, J,H,L: Immunology at a Glance 5th ed. Black well Scientific Publications. London, 1992).

Macrophage is activated into an activated macrophage when the macrophage is exposed to bacteria or exogenous stimulators. The activated macrophage shows functional changes, such as phagocytosis, increase of protein synthesis by various enzymes such as increased prostaglandin secretion, etc., and increased cell size and cell secretion. Especially, it has been known that cytokines (IL-1β, IL-6, TNF-α), hydrogen peroxide (H₂O₂), nitrous acid (NO), cytolytic protease and like, which are secreted by the activated macrophage in mechanism of cytotoxic action on cancer cells, show cytotoxicity in cancer cells (Hibbs J. B. et al., Biochem. Biophys. Res. Commun., 157: 87-94, 1998).

The activated macrophage has an ability to facilitate an antimicrobial action and an anticancer action, but does not have an enough efficiency to facilitate an in vivo anticancer action in cancer patients when it is used alone. Anticancer therapies, including conventional chemotherapies, radiotherapies, etc., cause systemic side effects such as severely high fever, diaphoresis, headache and emesis. Accordingly, it is very important to develop the anticancer treatment using the immunostimulating activity.

It has been known that various biochemical mechanisms are involved in immuno regulation, in particular an enzyme nitric oxide synthase (NOS) producing nitric oxide (NO), and enzymes related to prostaglandin biosynthesis play an important role in the immunoregulation. Accordingly, the enzyme NOS producing NO from L-arginine, and the enzyme cyclooxygenase (COX) involved in synthesizing prostaglandins from arachidonic acid has been considered to be an important criterion of the immunoregulation (Chihara G. et al., Immunology, 34:695-711, 1978). The NOS and COX-2 are expressed through complex cell transmission mechanism, and various kinases, which transmit extern al signals into cells, take part in the cell transmission mechanism. In particular, expressions of nuclear factor-kappa B (NF-κB), iNOS and COX-2 are mainly affected. Tyrosine or serine/threonine kinases are phosphorylated and activated in response to stimuli of LPS, INF-α or like, and inhibitor-kappa B (I-κB), which is an inhibitor component of a NF-κB complex present in cytoplasm, is phosphorylated and activated by I-κB kinase, and therefore NF-κB is activated by degrading the protein I-κB (D'Acquisto F. et al., Mol. Interv. 2: 22-35, 2002). A transcription factor NF-κB is a sequence-specific DNA binding protein, one of important factors that induce various gene transcriptions which take part in cell growth, differentiation and immune response.

Accordingly, there have been ardent attempts to find natural substances capable of activating macrophage without any side effect, and it has been found that the natural substances have an activity in high molecular weight fractions rather than their low molecular weight fractions. Immunostimulators derived from the natural substances may be used to treat cancers, immune deficiency syndromes, chronic infections, etc. by strengthening immune responses or restoring weakened immune functions. There have been many studies on basidiomycetes, fungi, medicinal herbs, etc. to obtain an immune response modifier from the conventional natural substances. In particular, polysaccharides have been mainly reported to be these components in a high molecular weight fraction, and anticancer activities, anti-complement activities and immunomodullating activities such as induced lymphocyte proliferation have been also found. Polysaccharides, for example lentinan, schzophyllan, bestatin, krestin and glucans such as peptide PSK, etc. have been mainly used for the anticancer treatment, and the polysaccharides having the immunomodullating activities are derived from fungi.

Meanwhile, Curcuma xanthorrhiza is a Zingiberaceae plant which is a traditional medicinal herb of Indonesia generally known as temu lawak or Javanese turmeric, and includes terpenoid-based compounds such as artumenone, α-curcumene, β-curcumene, curzerenone, germacrone, β-sesquiphellandrene, α-turmerone, β-turmerone, xanthorrhizol, etc., 7-30% of essential oil, 30-40% of carbohydrate, and 0.02-2.0% of aromatic pigments such as curcuminoid, etc. (Lin S. C. et al., Am. J. Chin. Med., 23:243-254, 1995).

### DISCLOSURE OF INVENTION

### Technical Problem

Accordingly, an object of the present invention to provide a substance that is safe since it is isolated from natural substances and also is excellent in immunostimulating and/or anticancer effects, a manufacturing method thereof, and a use thereof.

### Technical Solution

In order to accomplish the above object, the present invention provides a method for manufacturing polysaccharides isolated from Curcuma xanthorrhiza, including step of: (S1) preparing a powder of Curcuma xanthorrhiza Roxb.; (S2) extracting the powder with an organic solvent, and then filtering or centrifuging to obtain a residue; (S3) extracting the residue to prepare a solution containing polysaccharides; (S4) removing starch by adding starch-hydrolyzing enzyme to the polysaccharides-containing solution; (S5) precipitating the polysaccharides after the step (S4); and (S6) purifying the polysaccharides after the step (S5).

Also, the present invention provides polysaccharides isolated from Curcuma xanthorrhiza obtained according to the manufacturing method, and an immunostimulating composition including the polysaccharides.

The inventors have tried to search for immunostimulators from various natural substances and found that polysaccharides isolated from Curcuma xanthorrhiza Roxb. have a good immunostimulating activity, and therefore the present invention was completed on the basis of the facts.

Hereinafter, the immunostimulating polysaccharides isolated from Curcuma xanthorrhiza of the present invention, the manufacturing method thereof, and an immunostimulating or anticancer composition comprising the polysaccharides will be described in detail.

The present invention provides a method for manufacturing polysaccharides isolated from Curcuma xanthorrhiza, including steps of (S1) preparing a powder of Curcuma xanthorrhiza Roxb.; (S2) extracting the powder with an organic solvent, and then filtering or centrifuging to obtain a residue; (S3) extracting the residue to prepare a solution containing polysarcharides; (S4) removing starch by adding starch-hydrolyzing enzyme to the polysaccharides-containing solution; (S5) precipitating the polysaccharides after the step (S4); and (S6) purifying the polysaccharides after the step (S5).

The manufacturing method of the present invention comprises a step of preparing a powder of Curcuma xanthorrhiza Roxb. (S1). The powder of Curcuma xanthorrhiz a Roxb. may be prepared according to conventional powdering methods in the art to which the present invention pertains.

The manufacturing method of the present invention comprises a step of extracting the powder with an organic solvent, and then filtering or centrifuging to obtain an insoluble residue (S2). The organic solvent includes, but is not limited to, methanol, ethanol, propanol, isopropanol, butanol, acetone, ether, benzene, chloroform, ethylacetate, methylene chloride, hexane, cyclohexane, petroleum ether, etc., and they may be used alone or in combinations thereof. More preferably, ethanol, methanol, hexane or mixtures thereof may be used herein.

The manufacturing method of the present invention comprises a step of extracting the residue to prepare a solution containing polysaccharides (S3). Preferably, the polysaccharide components included in the residue may be obtained by extracting residues with hot water, an acid solution or an alkaline solution.

A purified water having a level of temperature at which the polysaccharides can be dissolved may be used as the hot water, and more preferably the purified water having a temperature of approximately 70 to 100° C. may be used herein.

Solutions, well known in the art to which the present invention pertains, having a suitable acidity for dissolving the polysaccharides may be used as the acid solution, an d, for example, at least one organic acid solution such as, but not limited to, citric acid, fumaric acid, lactic acid, tartaric acid, succinic acid, maleic acid, malic acid, oxalic acid, aspartic acid, glutamic acid, palmitic acid, propionic acid, ascorbic acid, chitoic acid, hippuric acid, alginic acid, cholic acid, butyric acid, benzoic acid, methanesulfonic acid, benzenesulfonic acid, toluenesulfonic acid, salicylic acid, gluconic acid, glycolic acid, mandelic acid, cinnamic acid, and/or at least one inorganic acid solution such as, but not limited to, hydrochloric acid, phosphoric acid, acetic acid, trifluoroacetic acid, hydrobromide, sulphuric acid may be used to make the acid solution. 0.005 to 10N HCl solution is preferably used in the terms of the manufacturing cost, etc., and 0.1 to 5 N HCl solution is more preferred.

Solutions, well known in the art to which the present invention pertains, having a suitable alkalinity for dissolving the polysaccharides may be used as the basic solution, and for example, at least one solution selected from the group consisting of, but is not limited to, sodium hydroxide, potassium hydroxide, sodium carbonate, calcium carbonate, sodium bicarbonate, potassium bicarbonate, pyridine, triethylamine and N,N-diisopropylethylamine. 0.005 to 10N NaOH solution is preferably used in the terms of the manufacturing cost, etc., and 0.1 to 5 N NaOH solution is more preferred.

The manufacturing method of the present invention comprises a step of removing starch by adding starch-hydrolyzing enzyme to the polysaccharides-containing solution (S4). Preferably, α-amylase, glucoamylase and the like may be used as the starch-hydrolyzing enzyme.

The manufacturing method of the present invention comprises a step of, after the step (S4), precipitating the polysaccharides (S5). More preferably, the polysaccharide may be precipitated from the starch-free polysaccharides-containing solution by adding a lower alcohol selected from the group consisting of methanol, ethanol, isopropanol, propanol, n-butanol, iso-butanol, tert-butanol, ethylene glycol, propylene glycol, glycerine, trimethylene glycol, etc.

The manufacturing method of the present invention comprises a step of, after the step (S5), purifying the polysaccharides (S6). The polysaccharides may be purified from a precipitated polysaccharide fraction by removing low molecular weight component using a molecular size fractionation system such as dialysis, ultra-filtration, etc. A membrane having a molecular weight cut off size of 500 to 10,000, preferably 500 to 5,000, more preferably 1,000 to 5,000 may be used for as dialysis, ultra-filtration, etc.

Also, the present invention provides polysaccharides isolated from Curcuma xanthorrhiza, obtained according to the manufacturing method, and a pharmaceutical composition comprising the polysaccharides as effective component. The polysaccharides isolated and purified from Curcuma xanthorrhiza according to the above procedure was tested on its immunostimulating activity. As a result, immunostimulating index like production amounts of NO, H₂O₂ and PGE₂, phagocytotic ability, and expressions of iNOS, TNF-α, COX-2 mRNA and proteins are increased. Also, the polysaccharides isolated from Curcuma xanthorrhiza exhibited an ability to kill cancer cells and an anticancer effect. Such an activity means that the polysaccharides isolated and purified from Curcuma xanthorrhiza according to the present invention may be effectively used as immuno stimulating composition and anticancer supplementary composition. That is, the polysaccharides according to the present invention may be very effectively used in drugs and functional foods for stimulating macrophage activity, preventing and treating immunological diseases including cancer, and enhancing immunity after the treatment of the immunological diseases.

Also, the immunostimulating composition according to the present invention may be used as effective preparation to treat diseases induced by immune depression, for example intractable diseases in the clinical immunology, chronic diseases, diabetes, cancer, male infertility, acquired immune deficiency syndrome (AIDS), pathological viral diseases, opportunistic infections, and diseases induced by the radiation exposure.

The composition comprising the polysaccharides, obtained according to the manufacturing method of the present invention may be manufactured in forms of medicinal drug and functional food according to the method as widely known to those skilled in the art to which the present invention pertains. The medicinal drug and the functional food may further include a pharmaceutically acceptable excipient or additive. The composition comprising the polysaccharides of the present invention may be administered alone or in combination with any of convenient carrier, excipient, etc., and be administered in a single dose or in divided doses.

The medicinal drug and the functional food comprising the polysaccharides of the present invention may be formulated in a solid or liquid form. The solid formulation includes, but is not limited to, a powder, a granule, a tablet, a capsule, a suppository, etc. Also, the solid formulation may further include, but is not limited to, an diluent, a flavoring agent, a binder, a preservative, a disintegrating agent, a lubricant, a filler, etc. The liquid formulation includes, but is not limited to, a solution such as water solution and propylene glycol solution, a suspension, an emulsion, etc., and may be prepared by adding suitable additives such as a coloring agent, a flavoring agent, a stabilizer, a thickener, etc.

For example, the power may be prepared by simply mixing a pharmaceutically acceptable excipient such as lactose, starch, microcrystalline cellulose and the like, with the polysaccharides of the present invention. The granule may be prepared by mixing a pharmaceutically acceptable excipient; and a pharmaceutically acceptable binder such as polyvinylpyrrolidone, hydroxypropylcellulose, etc. with the polysaccharides of the present invention, and then undergoing a wet granulation process using a solvent such as water, ethanol, isopropanol, etc. or a dry granulation process using compressive force. Also, the tablet may be prepared by mixing the granule with a pharmaceutically acceptable lubricant such as magnesium stearate, and then tabletting the resultant mixture using a tablet machine.

The composition of the present invention may be administered in forms of, but not limited to, oral, injectable, inhalable, intranasal, vaginal, rectal, sublingual, etc. depending on the disorders to be treated and the patient's conditions. The composition of the present invention may be formulated in a suitable dosage unit comprising a pharmaceutically acceptable and non-toxic carrier, additive and/or vehicle, which all are generally used in the art, depending on the routes to be administered.

The polysaccharides of the present invention may be administered daily at a dose of approximately 0.2 to approximately 200 mg/kg, preferably approximately 2 to approximately 50 mg/kg, more preferably approximately 5 to approximately 30 mg/kg. How ever, the dosage may be varied according to the patient's conditions (age, sex, body weight, etc.), the severity of patients in need thereof, the used effective components, diets, etc. The polysaccharides of the present invention may be administered once or several times per day in divided doses, if necessary.

The present invention provides a method for supplementing anticancer drug or stimulating immune system, comprising administering the polysaccharides according to the present invention as effective component.

A toxicity test was conducted by orally administering the polysaccharides of the present invention to rats, and as a result, it might be confirmed that the polysaccharides according to the present invention is very safe since 50% lethal dose (LD₅₀) in the oral toxicity test is 2,000 mg/kg or more.

### BRIEF DESCRIPTION OF THE DRAWINGS

These and other features, aspects, and advantages of preferred embodiments of the present invention will be more fully described in the following detailed description, taken accompanying drawings. In the drawings:

FIG. 1 is a flow chart showing that immunostimulating polysaccharides are isolated from Curcuma xanthorrhiza.

FIG. 2 is a diagram showing results obtained through gel permeation chromatography performed to determine a molecular weight of the polysaccharides isolated from Curcuma xanthorrhiza.

A: Gel permeation chromatography of pullulan used as reference material (Molecular Weight=788000, 112000, 22800, 5900, 360)

B: Gel permeation chromatography of the polysaccharides isolated from Curcuma xanthorrhiza

FIG. 3 is a diagram showing results obtained through Bio-LC performed to deter mine a sugar content of the polysaccharides isolated from Curcuma xanthorrhiza of the present invention.

A: Chromatography of glucose, arabinose, galactose, mannose, rhamnose and xy lose, which all used as reference material

B: Chromatography showing the sugar content of the polysaccharides isolated from Curcuma xanthorrhiza

FIG. 4 is a graph showing an effect of the polysaccharides isolated from Curcuma xanthorrhiza on increase in NO production.

FIG. 5 is a graph showing an effect of the polysaccharides isolated from Curcuma xanthorrhiza on increase in H₂O₂ production.

FIG. 6 is a graph showing an effect of the polysaccharides isolated from Curcuma xanthorrhiza on increase in phagocytotic ability.

A: Untreated group

B: Group treated with 30 ug/ml of sample

FIG. 7 is a graph showing an effect of the polysaccharides isolated from Curcuma xanthorrhiza on increase in TNF-α protein expression.

FIG. 8 is a graph showing an effect of the polysaccharides isolated from Curcuma xanthorrhiza on increase in TNF-α mRNA expression.

FIG. 9 is a graph showing an effect of the polysaccharides isolated from Curcuma xanthorrhiza on increase in iNOS protein expression.

FIG. 10 is a graph showing an effect of the polysaccharides isolated from Curcuma xanthorrhiza on increase in iNOS mRNA expression.

FIG. 11 is a graph showing an effect of the polysaccharides isolated from Curcuma xanthorrhiza on increase in COX-2 protein expression.

FIG. 12 is a graph showing an effect of the polysaccharides isolated from Curcuma xanthorrhiza on increase in COX-2 mRNA expression.

FIG. 13 is a graph showing IκBα phosphorylation of the polysaccharides isolated from Curcuma xanthorrhiza.

FIG. 14 is a graph showing an effect of the polysaccharides isolated from Curcuma xanthorrhiza on increase in in vivo NO production.

FIG. 15 is a graph showing an effect of the polysaccharides isolated from Curcuma xanthorrhiza on increase in in vivo phagocytotic ability.

FIG. 16 is a graph showing an effect of the polysaccharides isolated from Curcuma xanthorrhiza on increase in in vivo ability to kill cancer cells.

FIG. 17 is a graph showing an effect of the polysaccharides isolated from Curcuma xanthorrhiza on increase in in vivo iNOS mRNA expression.

FIG. 18 is a graph showing an effect of the polysaccharides isolated from Curcuma xanthorrhiza on increase in in vivo TNF-α mRNA expression.

FIG. 19 is a graph showing an effect of the polysaccharides isolated from Curcuma xanthorrhiza on increase in in vivo IL-1β mRNA expression.

FIG. 20 is a graph showing an effect of the polysaccharides isolated from Curcuma xanthorrhiza on increase in in vivo IL-6 mRNA expression.

### MODES FOR CARRYING OUT THE INVENTION

Hereinafter, preferred embodiments of the present invention will be described in detail referring to the accompanying drawings. However, the description proposed herein is just a preferable example for the purpose of illustrations only, not intended to limit the scope of the invention, so it should be understood that other equivalents and modifications could be made thereto without departing from the spirit and scope of the invention.

Hereinafter, all test results were obtained by conducting the activity analysis at least three times, and the results are represented by mean±standard deviation. The statistical analysis was conducted using a Duncan test (SPSS 12.0), and proven to be statistically significant if a value *p is 0.05 or less and a value **p is 0.01 or less.

### Example 1

### Isolation of Polysaccharides from Curcuma xanthorrhiza

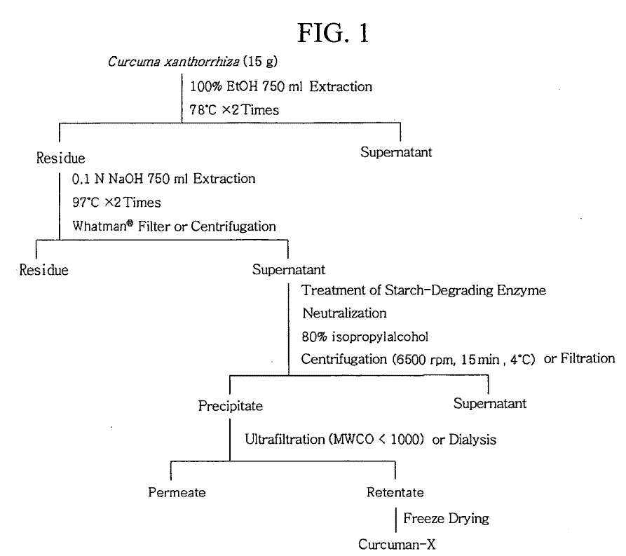

750 ml of 100% ethanol was added to 15 g of a powder of Curcuma xanthorrhiza Roxb. and extracted two times for 2 hours at 78° C. A supernatant and a residue were separated from the resultant extract using a Whatman filter (No. 2). 750 ml of an extraction solvent, 0.1 N NaOH, was added to the residue, and then extracted two times for 2 hours at 97° C. In order to hydrolize starch in the resultant 0.1 N NaOH extract, the extract was treated with α-amylase (Termainyl 120L, NOVO Nordisk A/S, Denmark) and glucoamylase (AMG 300L, NOVO Nordisk A/S, Denmark) under optimal enzyme conditions, and then neutralized. 4 times volume of isopropyl alcohol was added to the remaining solution, kept for 24 hours at 4° C. to precipitate polysaccharides, and then centrifuged for 15 minutes at 6,500 rpm to isolate the polysaccharides from a supernatant. The isolated precipitate was dissolved in purified water to make a final 1% solution, an d then subjected to ultra-filtration (thin channel ultrafiltration system, Amicon TCF-10, Amicon Co., U.S.A.) using a membrane having a molecular weight cut off (MWCO) of 1,000. After the ultra-filtration, solutions containing the precipitate having a molecular weight of 1,000 or more were collected and freeze-dried to obtain polysaccharides, which had a yield of 6%. The resultant polysaccharides were named “Curcuman-X” hereinafter. The entire extraction and isolation process according to one embodiment of the present invention was shown in FIG. 1.

### Experimental Example 1

### Molecular Weight Measurement

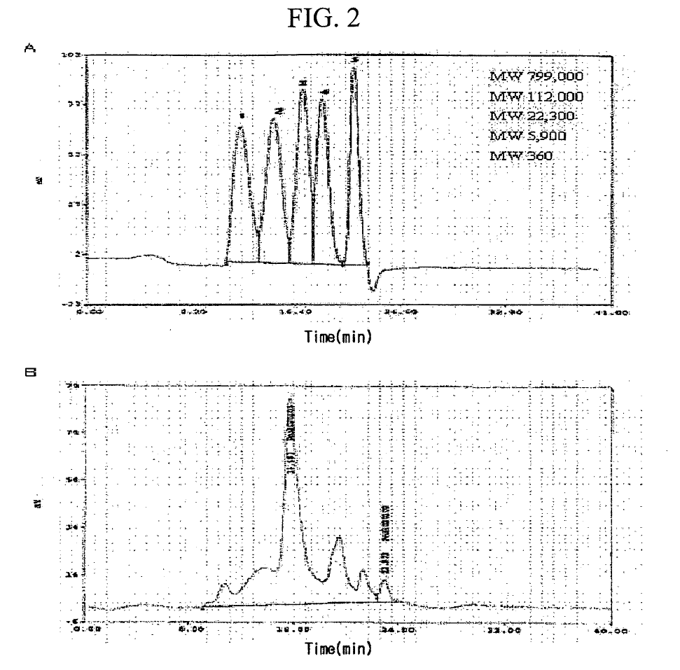

A molecular weight of Curcuman-X, the polysaccharides isolated from Curcuma xanthorrhiza in the Example 1, was measured using gel permeation chromatography. A used column was an Ultrahydrogel linear column and an Ultrahydrogel 500 column, and 0.1N NaNO₂ was used for a mobile phase. A flow rate of the mobile phase was 1 ml/min in its analysis, and pullulan was used as a reference material. The experimental results were shown in FIG. 2. As shown in FIG. 2, it was revealed that a number aver age molecular weight of the Curcuman-X is 33,000 Da.

### Experimental Example 2

### Measurement of Component Sugar

A component sugar content of Curcuman-X, the polysaccharides isolated from Curcuma xanthorrhiza in the Example 1, was measured using Bio-LC (Dionex DX-500, USA). 100 ul of 24 N sulfuric acid was added to 10 mg of the polysaccharides, reacted for 1 hour, and then the resultant mixture was filled with nitrogen and hydrolyzed for 3 hours at 100° C. After the reactant was cooled to room temperature, the cooled reactant was neutralized with 12N ammonium hydroxide and diluted with distilled water. The diluted solution was filtered with a filter, and then sugar content was measured using Bio-LC. As analysis conditions of the Bio-LC, a used column was CarboPac™ PA1, 2 2.6 mM NaOH was used as an isocratic eluent, and 200 mM NaOH was used as a regeneration buffer. A flow rate of the isocratic eluent was 0.3 ml/min, and an amount of added sample was 50 ul, in which the addition of the sample was conducted under a nitrogen gas atmosphere. A sugar content was checked according to retention times using glucose, galactose, arabinose, mannose, xylose and rhamnose as sugar reference materials.

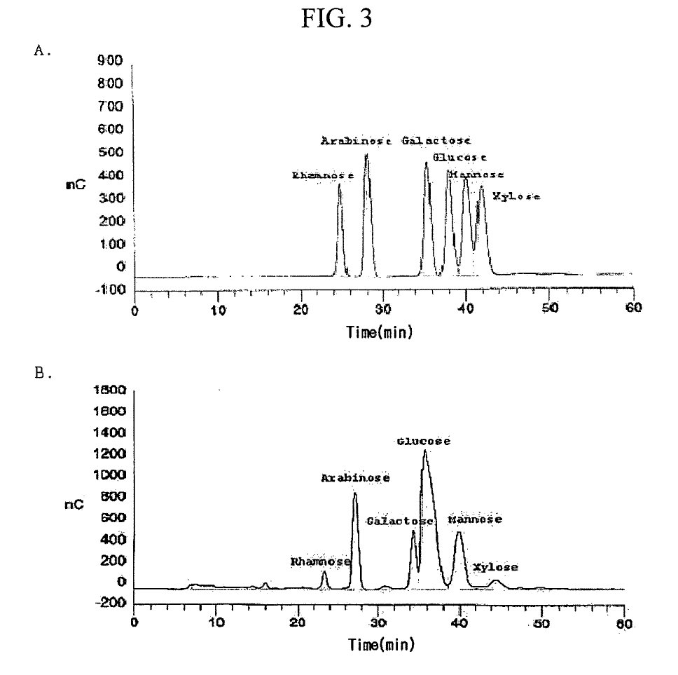

The measurement results of sugar content of the polysaccharides are shown in FIG. 3, and the component sugar contents are listed in the following Table 1. As listed in Table 1, the polysaccharides isolated from Curcuma xanthorrhiza are mainly composed of glucose, arabinose, galactose and mannose.

TABLE 1

| Major Sugar Component | Content |
|---|---|
| Glucose | 50.67% |
| Arabinose | 18.69% |
| Galactose | 14.0% |
| Mannose | 12.97% |
| Rhamnose | 2.73% |
| Xylose | 0.92% |

### Experimental Example 3

### Measurement of NO Production

In order to examine a correlation between an immune regulatory effect of the polysaccharides isolated in Example 1 and NO secretion, an ability to produce NO was measured using RAW264.7 macrophage. Murine macrophage cell line RAW264.7 cell was cultured in a complete medium, Dulbecco's Modified Eagles Medium, supplemented with 10% fetal calf serum, 100 units/ml penicillin and 100 ug/ml streptomycin, at 37° C. in a CO₂ incubator.

The RAW264.7 macrophage was divided at a density of 2×10⁵ cells/ml, cultured at 37° C. for 4 hours in a CO₂ incubator, and then treated with increasing densities (5, 10, 30, 50 ug/ml) of Curcuman-X and 10 ug/ml of lipopolysaccharide as control, and cultured for 24 hours. After the culture, a concentration of nitrite (a stable aqueous compound of NO) in the cultured supernatant was measured using a Griess assay (Griess. P., Chem. Ber. 12:426-428, 1897). That is to say, NaNO₂ was used as reference material an d a Griess reagent (0.5% sulfanilyamide, 0.05% N-(1-naphthyl)ethylene diamine dihydrochloride/2.5% H₃PO₄) was used to measure absorbance of the samples at a wavelength of 540 nm.

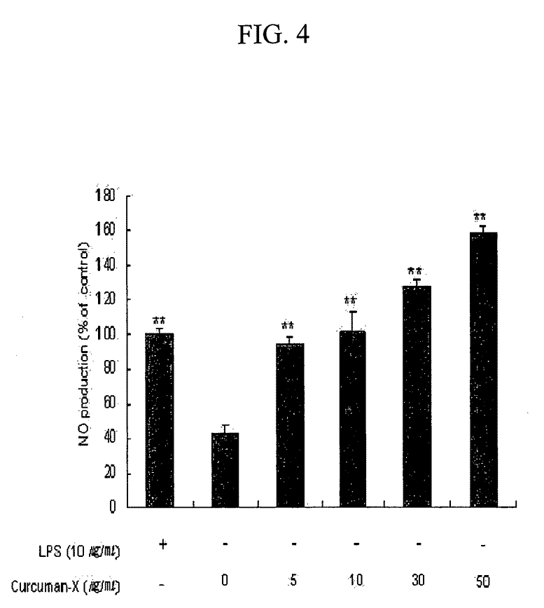

As a result, it was revealed that the NO production is significantly higher in the Curcuman-X-treated group than the untreated group and its values were increased in a d ose-dependent manner, as shown in FIG. 4, which indicates that the polysaccharides isolated from Curcuma xanthorrhiza highly increase the ability to produce NO of the macro phage.

### Experimental Example 4

### Measurement of H₂O₂ Production

In order to examine a correlation between an immune regulatory effect of the polysaccharides isolated in Example 1 and H₂O₂ secretion, an ability to produce H₂O₂ was measured using RAW264.7 macrophage. Murine macrophage cell line RAW264.7 cell was cultured in a complete medium, Dulbecco's Modified Eagles Medium, supplemented with 10% fetal calf serum, 100 units/ml penicillin and 100 ug/ml streptomycin, at 37° C. in a CO₂ incubator.

The hydrogen peroxide production was measured by using chromogenic reaction of horseradish peroxidase (HRP)-dependent oxidation process of phenol red with an Amplex Red reagent (10-acetyl-3,7-dihydroxyphenoxazi).

The RAW264.7 macrophage was divided at a density of 2×10⁴ cells/ml, treated with 50 mM Amplex Red reagent and 0.1 U/ml HRP in Krebs-Ringer phosphate (KRPG: 145 mM NaCl, 5.7 mM sodium phosphate, 4.86 mM KCl, 0.54 mM CaCl, 1.22 mM MgSO₄, 5.5 mM glucose, pH 7.35), and then treated with increasing densities (5, 10, 30, 50 ug/ml) of Curcuman-X and 10 ug/ml of lipopolysaccharide as control, and cultured for 20 hours. After the culture, a concentration of H₂O₂ in the cultured supernatant was measured by measuring absorbance of the samples at a wavelength of 590 nm.

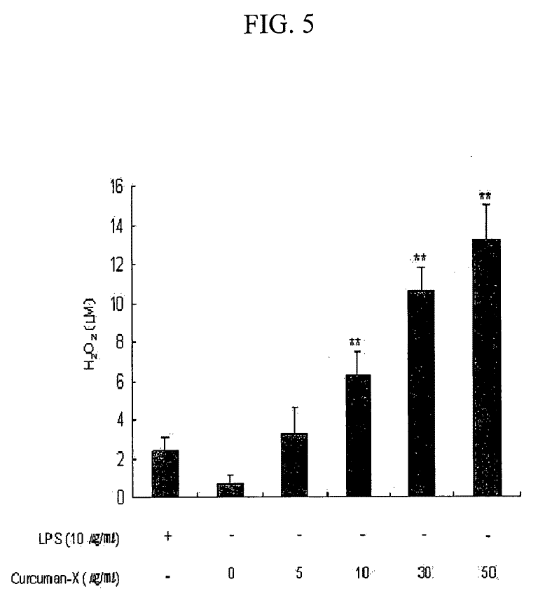

As a result, it was revealed that the H₂O₂ production is significantly higher in the Curcuman-X-treated group than the untreated group and its values were increased in a dose-dependent manner, as shown in FIG. 5. It was revealed that the RAW264.7 macrophage treated with 50 ug/ml of the polysaccharides exhibits a higher H₂O₂ production a much as 12 times compared to the untreated group, and their effect is more excellent than LPS used as control, which indicates that the polysaccharides isolated from Curcuma xanthorrhiza are substances having an activity of mitogen that highly increases the ability to produce H₂O₂ of the macrophage, and the increase of the H₂O₂ production of the macrophage by the polysaccharides plays an important role in adjacent cells in addition to destruction of bacteria invaded from the outside.

### Experimental Example 5

### Measurement of Phagocytotic Ability of Macrophage

The phagocytotic ability of the polysaccharides isolated in Example 1 was assessed with RAW264.7 macrophage, and measured by using heat-killed fluorescein isothiocyanate (FITC)-labeled Escherichia coli BioParticles (K-12 strain, Molecular Probes, Eu gene, OR, US). Murine macrophage cell line RAW264.7 cell was cultured in a complete medium, Dulbecco's Modified Eagles Medium, supplemented with 10% fetal calf serum, 100 units/ml penicillin and 100 ug/ml streptomycin, at 37° C. in a CO₂ incubator.

The RAW264.7 macrophage was divided at a density of 2×10⁵ cells/ml into a 96-well plate, treated with increasing densities (5, 10, 30, 50 ug/ml) of Curcuman-X, and then cultured at 37° C. in a CO₂ incubator. 4 hours after the culture, the heat-killed fluorescein isothiocyanate (FITC)-labeled Escherichia coli BioParticles was divided at a dose of 100 ul, and then cultured for 2 hours. After the culture, the macrophage and the bacteria were washed with PBS, trypan blue was divided at a dose of 100 ul, kept for 1 hour at room temperature, and then removed off to measure phagocytotic ability using a fluorescence emitter.

Also, the effect of Curcuman-X on phagocytotic activity of the activated macrophage was assessed using a confocal microscope (×1890).

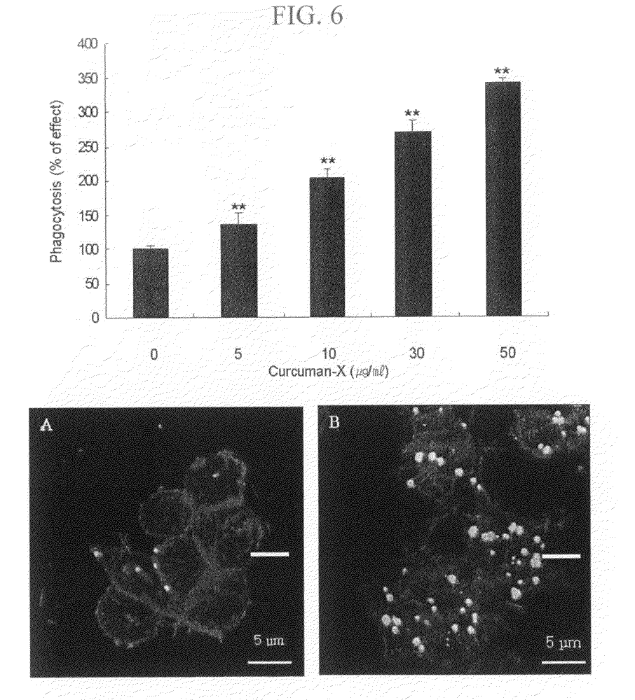

As a result, it was revealed that the phagocytotic ability is significantly higher in the Curcuman-X-treated group than the untreated group and its values were increased in a dose-dependent manner, as shown in FIG. 6. In the graph of FIG. 6, “A” represents a n untreated group, and “B” represents 30 ug/ml of Curcuman-X. Since the macrophage has different receptors that may recognize foreign substances, foods and natural substances may be directly associated with activation of the macrophage, but the macrophage may be activated by means of secondary actions by activity of complements or other lymphocytes. Therefore, an exact mechanism in which the polysaccharides isolated from Curcuma xanthorrhiza activate the macrophage was unknown, but the general immune system including acquired immunity and innate immunity may be strengthened by significantly improving the phagocytotic ability by means of the activated macrophage.

### Experimental Example 6

### Measurement of PGE₂ Production

An effect of Curcuman-X, the polysaccharides isolated in Example 1, on PGE₂ production was assessed with RAW264.7 macrophage, and the PGE₂ production was quantified using a R&D kit (R&D systems, USA). Murine macrophage cell line RAW264.7 cell was cultured in a complete medium, Dulbecco's Modified Eagles Medium, supplemented with 10% fetal calf serum, 100 units/ml penicillin and 100 ug/ml streptomycin, at 37° C. in a CO₂ incubator.

The RAW264.7 macrophage was divided at a density of 2×10⁵ cells/ml into a 96-well plate, stabilized at 37° C. for 4 hours in a CO₂ incubator, and then treated with increasing densities (5, 10, 30, 50 ug/ml) of Curcuman-X and 10 ug/ml of lipopolysaccharide as control, and then cultured for 24 hours. After the culture, a supernatant was transferred to a new well plate, and then 100 ul assay buffer, 50 ul PGE₂ conjugate buffer and a PGE₂ antibody solution were added to each well. The above-mentioned treated plate was reacted for 2 hours at room temperature. Reaction reagents was completely removed from the well plate, each well was washed with a washing solution, and then 50 ul P GE₂ conjugate buffer and 200 ul pNPP substrate were added to each well and reacted for 1 hour at room temperature. 50 ul reaction stopping solution was added to each well to stop the reaction, and then absorbance of the samples were measured at a wavelength of 405 nm.

The PGE₂ production was measured, and, as a result, it was revealed that the PG E₂ production is significantly higher in the Curcuman-X-treated group than the untreated group and its values were increased in a dose-dependent manner, as listed in Table 2. It was also revealed that the PGE₂ production is as much as 300% in the group treated with 50 ug/ml of the polysaccharides when compared to the untreated group, and their effect is more excellent than those of LPS used as control, which indicates that Curcuman-X, the polysaccharides isolated from Curcuma xanthorrhiza, significantly improves the PGE₂ production of the macrophage.

TABLE 2

| Sample | PGE₂ (ng/ml) | Untreated Group (%) |
|---|---|---|
| Untreated Group | 114.51 ± 3.81 | 100 |
| Control (LPS 10 ug/ml) | 331.16 ± 1.34* | 290.11 |
| Polysaccharides 5 ug/ml | 324.64 ± 0.92* | 284.64 |
| Polysaccharides 10 ug/ml | 346.64 ± 1.94* | 303.67 |
| Polysaccharides 30 ug/ml | 360.02 ± 3.93* | 315.39 |
| Polysaccharides 50 ug/ml | 366.40 ± 0.48* | 320.98 |

### Experimental Example 7

### Effect on Secretion of iNOS, TNF-α and COX-2

In order to assess an effect of Curcuman-X isolated in Example 1 on protein and mRNA expression of iNOS, TNF-α and COX-2, Western blot and RT-PCR were conducted. Murine macrophage cell line RAW264.7 cell (Korean Cell Line Bank) was cultured in a complete medium, Dulbecco's Modified Eagles Medium, supplemented with 10% fetal calf serum, 100 units/ml penicillin and 100 ug/ml streptomycin, at 37° C. in a CO₂ incubator.

The RAW264.7 cell was adjusted to 2×10⁶ cells/ml, divided into a 60 mm culture vessel, and culture for 6 hours to obtain cells for Western blotting. The cultured cells were treated with increasing densities (5, 10, 30, 50 ug/ml) of Curcuman-X dissolved in DPBS (Dulbecco's phosphate buffered saline) and 10 ug/ml of the lipopolysaccharide as control. 24 hours after each of the samples was treated, the medium was removed from the culture vessel, and the culture vessel was washed twice with DPBS solution, and then 1 ml of DPBS was added to the culture vessel, the cell solution was collected and centrifuged (1,500 rpm, 3 mins) to collect cells. In order to obtain proteins of the collected cells, 100 ul of lysis buffer (200 mM Tris, 150 mM NaCl, 1 mM EDTA, 1 mM EGTA, 1% triton, 1 mM PMSF, protease inhibitor cocktail) was added to lyse the cells, and then proteins were collected. The collected proteins were quantified using a Bradford assay. At this time, bovine serum albumin was used as reference standard. The extracted proteins were electrophoresized in a 10% SDS-polyacrylamide gel, and then the proteins in the gel were transferred to a nitrocellulose membrane. The membrane was blocked with 5% skim milk for 1 hour at room temperature so as to prevent the membrane from being contaminated by unknown proteins any more, and primary antibodies of iNOS, TNF-α and COX-2 were diluted with a blocking solution at a ratio of 1:1000 and reacted for 2 hours at room temperature. After the reaction with the primary antibodies, the membrane was washed 3 times with TBST (Tris-buffer Saline Tween 20) with shaking for 10 minutes. Secondary antibodies recognizing the primary antibodies of iNOS, TNF-α and COX-2 were diluted with 5% skim milk to a ratio of 1:2000, reacted for 1 hour at room temperature, washed 3 times with TBST (Tris-buffer Saline Tween 20) with shaking for 10 minutes in the same manner as in the primary antibodies, and then developed using chemiluminescence.

In order to conduct RT-PCR, RAW264.7 cells were divided into a 60 mm cell culture dish at a dose of 2×10⁶ cells/ml, and then stabilized overnight. The cells was treated with the sample, collected and washed with PBS, and then 1 ml of triazole (Invitrogen, USA) was added and stirred at room temperature. 200 ul of chloroform was added and stirred again, and the cell mixture was centrifuged at 12,000 rpm for 15 minutes at 4° C., isopropyl alcohol was added to supernatant, and then the cell mixture was centrifuged again to obtain an RNA pellet. The RNA obtained thus was transcribed into cDNA using MMLV reverse transcriptase. Primers of iNOS (sense 5′-CAACCAGTATTATGGCTCCT-3′, antisense 5′-GTGACAGCCCGGTCTTTCCA-3′), TNF-α (sense: 5-CCTGTAGCCCACGTCGTAGC-3, antisense: 5-T7GACCTCAGCGCTGAGTTG-3), COX-2 (sense 5′-CCGTGGTAATGTATGAGCA, antisense 5′-CTCGCTTCTGATATGTCTT-3′) and β-actin (sense 5′-TGGAATCCTGTGGCATCCATGAAAC-3′, antisense 5′-TAAAACGCAGCTCAGTAACAGTCCG-3′) were added thereto, and then each gene was amplified using Taq polymerase. At this time, a gene amplification condition is 30 cycles of: 30 sec. at 95° C., 1 min. at 55° C. and 1 min. at 72° C., and then one cycle of 5 min. at 72° C. The amplified cDNA was electrophoresized in a 1% agarose gel, and observed using a UV detector.

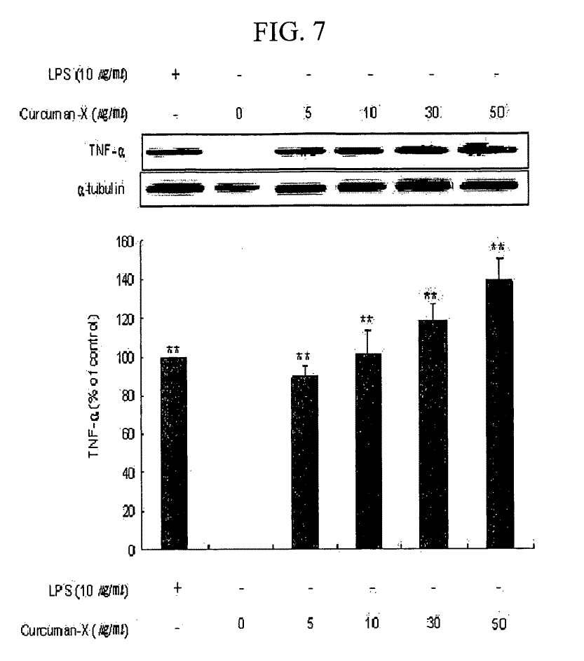

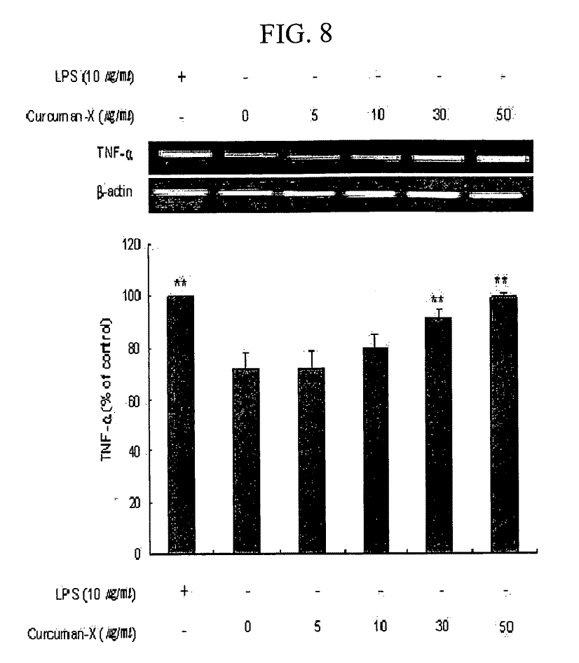

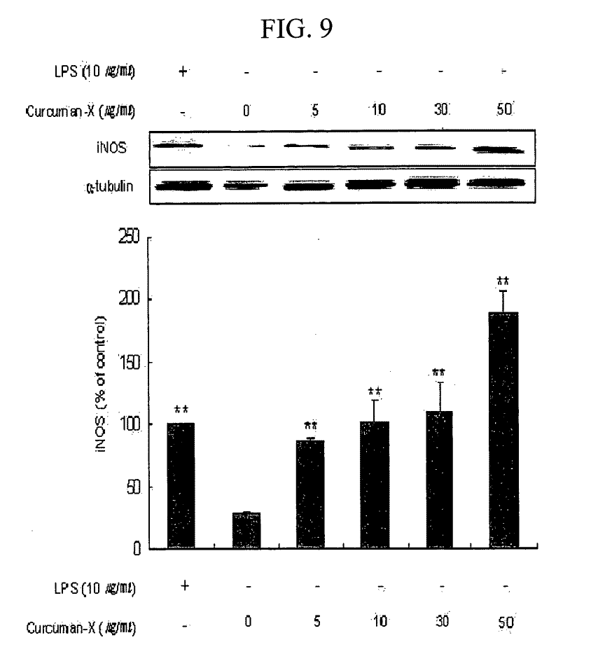

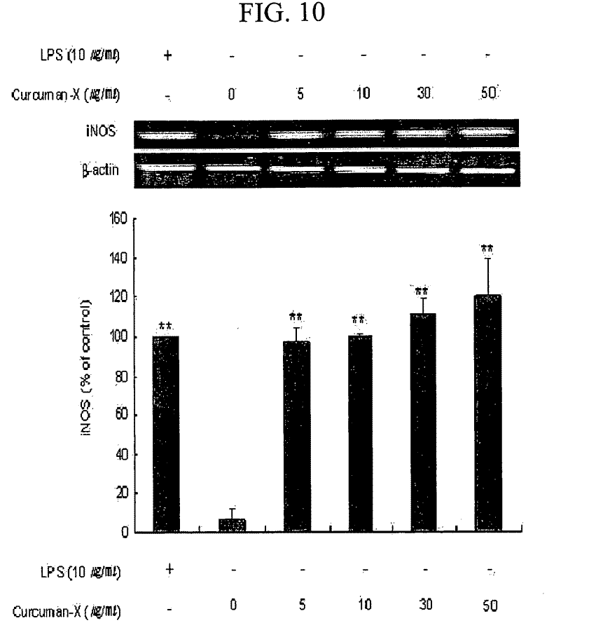

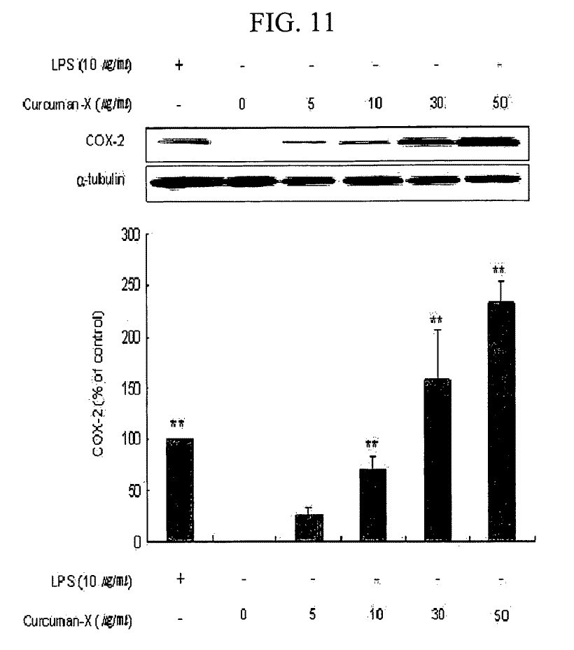

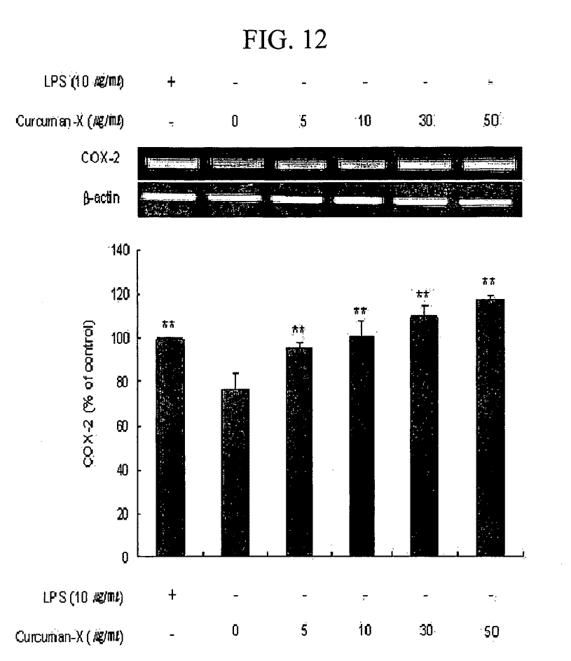

As a result, it was revealed that expressions of the iNOS, TNF-α and COX-2 proteins are clearly increased by Curcuman-X, as shown in FIG. 7, FIG. 9 and FIG. 11, and their mRNAs are also increased at the similar levels to those of the proteins, as shown in FIG. 8, FIG. 10 and FIG. 12. The results indicate that the increases of NO and PGE₂ as described in the Experimental examples are caused by the control of the mRNA and protein expressions.

### Experimental Example 8

### Measurement of IκBα Phosphorylation

In order to determine an effect of Curcuman-X isolated in Example 1 on IκBα phosphorylation, a Western blot was carried out. Murine macrophage cell line RAW264. 7 cell (Korean Cell Line Bank) was cultured in a complete medium, Dulbecco's Modified Eagles Medium, supplemented with 10% fetal calf serum, 100 units/ml penicillin and 100 ug/ml streptomycin, at 37° C. in a CO₂ incubator.

The RAW264.7 cell was adjusted to 2×10⁶ cells/ml, divided into a 60 mm culture vessel, and cultured for 6 hours to obtain cells for Western blotting. The cultured cells were treated with increasing densities (5, 10, 30, 50 ug/ml) of Curcuman-X dissolved in DPBS (Dulbecco's phosphate buffered saline) and 10 ug/ml of the lipopolysaccharide as control. 24 hours after each of the samples was treated, the medium was removed from the culture vessel, and the culture vessel was washed twice with a DPBS solution, and then 1 ml of DPBS was added to the culture vessel, the cell solution was collected and centrifuged (1,500 rpm, 3 min) to collect cells. In order to obtain proteins of the collected cells, 100 ul of lysis buffer (200 mM tris, 150 mM NaCl, 1 mM EDTA, 1 mM EGTA, 1% triton, 1 mM PMSF, protease inhibitor cocktail) was added to lyse the cells, and then proteins were collected. The collected proteins were quantified using a Bradford assay. At this time, bovine serum albumin was used as reference standard. The extracted proteins were electrophoresized in a 10% SDS-polyacrylamide gel, and then the proteins in the gel were transferred to a nitrocellulose membrane. The membrane was blocked with 5% skim milk for 1 hour at a room temperature so as to prevent the membrane from being contaminated by unknown proteins any more, and a primary antibody of pIκBα were diluted with a blocking solution at a ratio of 1:1000 and reacted for 2 hours at a room temperature. After the reaction with the primary antibody, the membrane was washed 3 times with TBST (Tris-buffer Saline Tween 20) while shaking for 10 minutes.

A secondary antibody recognizing the primary antibody was diluted with 5% skim milk to a ratio of 1:2000, reacted for 1 hour at a room temperature, washed 3 times with TBST (Tris-buffer Saline Tween 20) while shaking for 10 minutes in the same manner as in the primary antibody, and then developed using chemiluminescence.

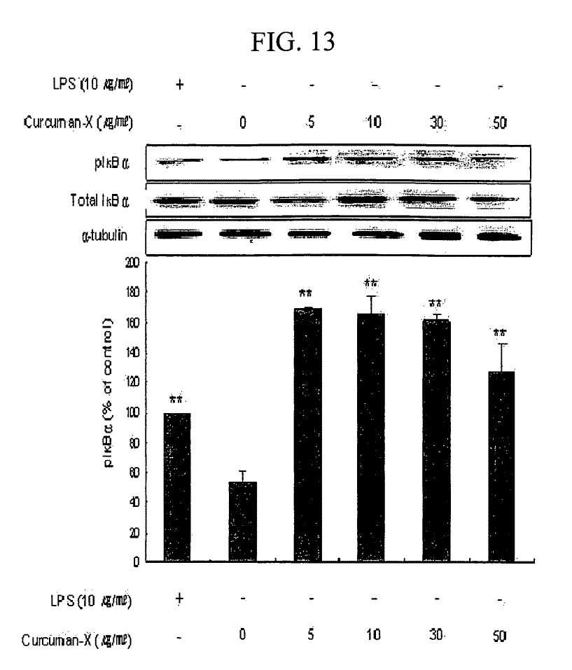

As a result, it was revealed that the IκBα protein is clearly phosphorylated by Curcuman-X, as shown in FIG. 13. The results indirectly indicate that the increases of iNOS, TNF-α and COX-2 as described in the Experimental examples are caused by the NF-κB activation.

### Experimental Example 10

### In vivo Measurement of NO Production by Oral Administration of Curcuman-X

In order to examine a correlation between an immune regulatory effect of Curcuman-X isolated in Example 1 and NO secretion, an ability to produce NO was observed using an animal experiment.

C57BL/6 mice (17-18 g, female) were divided into three groups, each group including 12 mice. Then, the mice in each of the groups were daily administered orally once with Curcuman-X at densities of 10, 50 and 100 mg/kg for 21 days. 2 ml of 3% thioglycollate medium was administered intraperitoneally, and, 3 days after the administration, intraperitoneal membrane was washed with 8 ml of a RPIM complete medium (including 10% fetal calf serum, 100 units/ml penicillin and 100 ug/ml streptomycin) to collect peritoneal macrophage, and the resultant peritoneal macrophage was attached to a FBS-coated dish for 4 hours, and then floating cells were removed off and the cell number of the resultant pure macrophage was counted.

The macrophage was divided at a density of 5×10⁵ cells/ml, cultured at 37° C. f or 24 hours in a CO₂ incubator. After the culture, nitrite in the cultured supernatant was measured and a Griess reagent (0.5% sulfanilyamide, 0.05% N-(1-naphthyl)ethylene diamine dihydrochloride/2.5% H₃PO₄) was used to measure absorbance of the samples at a wavelength of 540 nm with a microplate reader.

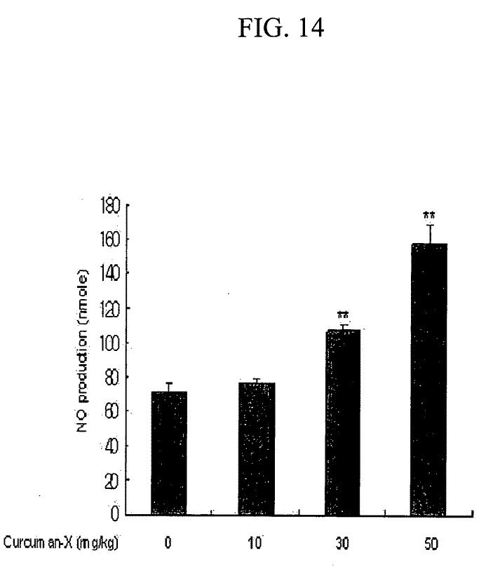

As a result, it was revealed that the NO production was increased when the mice were administered with Curcuman-X obtained in Example 1, as shown in FIG. 14, which indicates that the polysaccharides were absorbed and showed immune regulatory effect in the mice.

### Experimental Example 11

### In Vivo Effect of Oral Administration of Curcuman-X on Phagocytotic Ability

C57BL/6 mice (17-18 g, female) were divided into three groups, each group including 12 mice. Then, the mice in each of the groups were daily administered orally once with Curcuman-X at densities of 10, 50 and 100 mg/kg for 21 days. 2 ml of 3% thioglycollate medium was administered intraperitoneally, and intraperitoneal membrane was washed with 8 ml of a RPIM complete medium (including 10% fetal calf serum, 100 units/ml penicillin and 100 ug/ml streptomycin) 8 ml to collect peritoneal macrophage, and the resultant peritoneal macrophage was attached to a FBS-coated dish for 4 hours, and then floating cells were removed off and the cell number of the resultant pure macrophage was counted.

The macrophage was divided at a density of 5×10⁵ cells/ml, cultured at 37° C. for 24 hours in a CO₂ incubator. 4 hours after the culture, the heat-killed fluorescein isothiocyanate (FITC)-labeled Escherichia coli BioParticles was divided at a dose of 100 ul, and then culture for 2 hours. After the culture, the macrophage and the bacteria were washed with PBS, and trypan blue was divided into the macrophage and the bacteria at a dose of 100 ul, kept for 1 minute at room temperature, and then removed off to measure phagocytotic ability using a fluorescence emitter.

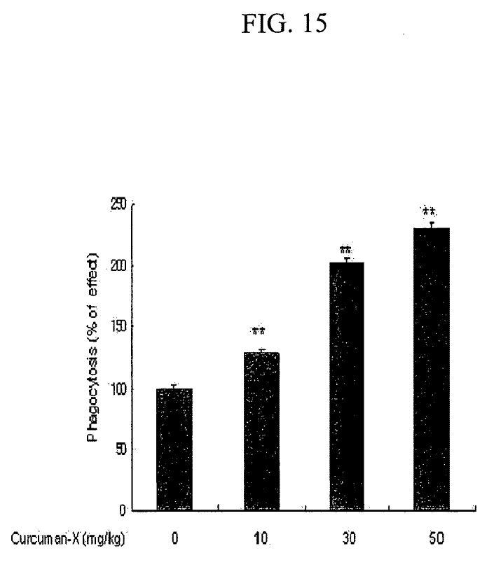

As a result, it was revealed that the phagocytotic ability is increased and its values were increased in a dose-dependent manner when the mice were administered with Curcuman-X obtained in Example 1, as shown in FIG. 15, which indicates that the polysaccharides isolated from Curcuma xanthorrhiza highly increase the in vivo phagocytotic ability of the macrophage.

### Experimental Example 12

### In vivo Induction of Spleen Cell Proliferation by Oral Administration of Curcuman-X

C57BL/6 mice (17-18 g, female) were divided into three groups, each group including 12 mice. Then, the mice in each of the groups were daily administered orally once with Curcuman-X at densities of 10, 50 and 100 mg/kg for 21 days. In order to determine growth of the spleen cell, the mice were sacrificed 21 days after the administration, spleen was taken out, and then spleen cell was extracted in a RPIM complete medium (including 10% fetal calf serum, 100 units/ml penicillin and 100 ug/ml streptomycin) u sing a slide glass. The extracted cell was divided at a dose of 2×10⁷ cells/ml and cultured at 37° C. for 72 hours in a CO₂ incubator. After the culture, an MTT solution (3-[4,5-dimethylthiazol-2-yl]-2,5-diphenyl-tetrazolium bromide) was added thereto and cultured for 4 hours. MTT-formazan products were dissolved by adding an equivalent volume of lysis buffer (DMSO). An amount of formazan was determined by measuring absorbance of the samples at a wavelength of 570 nm using a microplate reader.

The experimental results are represented by ratios of spleen cells of the mice treated with Curcuman-X to spleen cells of the untreated mice, as listed in the following Table 3.

TABLE 3

| Dose | No. of Spleen Cell (% based on Control) |
|---|---|
| 0 mg/kg | 100 ± 8.71 |
| Curcuman-X 10 mg/kg | 148.15 ± 12.25 |
| Curcuman-X 50 mg/kg | 189.2 ± 11.99 |
| Curcuman-X 100 mg/kg | 246.65 ± 10.63 |

As listed in Table 3, it was revealed that Curcuman-X of the present invention in creases the number of the spleen cells in a dose-dependent manner, and therefore has an immunostimulating effect since the increase of the spleen cells may become an immuno stimulation index.

### Experimental Example 13

### In vivo Measurement for Spleen Cell to Kill Cancer Cell by Oral Administration of Curcuman-X

In recent years, anticancer treatments using an immunostimulating agent have be en widely used and developed since many side effects are caused in cancer treatments using anticancer chemotherapic drugs. The use of the immunostimulating agent may mitigate the side effects caused by the anticancer drugs, and also improve the effect on the anticancer treatment. In the Examples, it was proven that Curcuman-X has the in vivo and ex vivo immunostimulating effects. In this Example, it was also proven that the immunostimulation by Curcuman-X has the anticancer effect.

C57BL/6 mice (17-18 g, female) were divided into three groups, each group including 12 mice. Then, the mice in each of the groups were daily administered orally once with the polysaccharides at densities of 10, 50 and 100 mg/kg for 21 days. In order to determine growth of the spleen cell, the mice were sacrificed 21 days after the administration, spleen was taken out, and then spleen cell was extracted in a RPIM complete medium (including 10% fetal calf serum, 100 units/ml penicillin and 100 ug/ml streptomycin) using a slide glass. The extracted cell was divided at a dose of 2×10⁶ cells/ml and cultured at 37° C. for 72 hours in a CO₂ incubator. An cancer cell YAC-1 cell was labeled with DiOC18 (3,3′-dioctadecyl oxacarbocyanine perchlorate, Molecular Probes, Eugene, U.S.A.) which produces a green fluorescent color. The cultured spleen cells and the labeled target cells (YAC-1 cells) was mixed at a ratio of 50:1 and cultured for 24 hours. After the culture, 10 ul of propidium iodide (PI, Sigma, U.S.A.) was added to the mixture, and then an ability for the spleen cells to kill the cancer cells was measured using a FACScan flow cytometer (Becton Dickinson, Heidelberg, German).

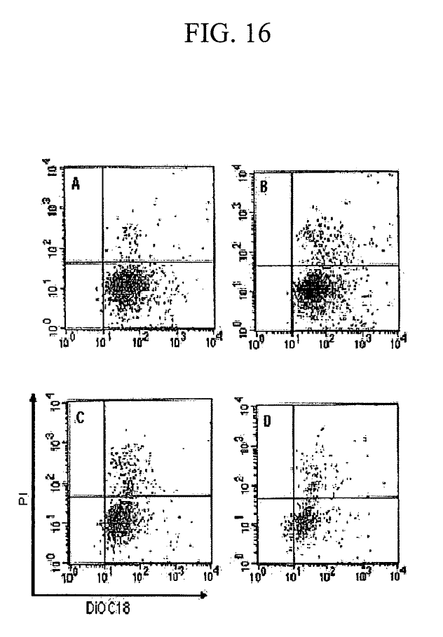

As a result, it was revealed that the ability for the activated spleen cells to kill the cancer cells is increased in a dose-dependent manner by the oral administration of Curcuman-X, as shown in FIG. 16, which indicates that Curcuman-X is the anticancer and immunostimulating agent showing the anticancer effect by enhancing the in vivo immunity rather than the cytotoxicity.

### Experimental Example 14

### In vivo Effect of Oral Administration of Curcuman-X on Cytokine Secretion

C57BL/6 mice (17-18 g, female) were divided into three groups, each group including 12 mice. Then, the mice in each of the groups were daily administered orally once with Curcuman-X at densities of 10, 50 and 100 mg/kg for 21 days. 2 ml of 3% thioglycollate medium was administered intraperitoneally, and, 3 days after the administration, intraperitoneal membrane was washed with 8 ml of a RPIM complete medium (including 10% fetal calf serum, 100 units/ml penicillin and 100 ug/ml streptomycin) to collect peritoneal macrophage, and the resultant peritoneal macrophage was attached to a FBS-coated dish for 4 hours, and then floating cells were removed off and the cell number of the resultant pure macrophage was counted.

The macrophage was divided at a dose of 5×10⁶ cells/ml, and then cultured at 37° C. for 24 hours in a CO₂ incubator. After the culture, the cells were collected, washed with PBS, and then 1 ml of triazole (Invitrogen, USA) was added and stirred at a room temperature. 200 ul of chloroform was added and stirred again, and the cell mixture was centrifuged at 12,000 rpm for 15 minutes at 4° C., isopropyl alcohol was added to supernatant, and then the cell mixture was centrifuged again to obtain an RNA pellet. The RNA obtained thus was transcribed into cDNA using MMLV reverse transcriptase. Primers of iNOS (sense 5′-CAACCAGTATTATGGCTCCT-3′, antisense 5′-GTGACAGCCCGGTCTTTCCA-3′), TNF-α (sense: 5-CCTGTAGCCCACGTCGTAGC-3, antisense: 5-TTGACCTCAGCGCTGAGTTG-3), IL-1 (sense: 5-TGCAGAGTTCCCCAACTGGTACATC-3, antisense: 5-GTGCTGCCTAATGTCCCCTTGAATC-3), IL-6 (sense: 5-GATGCTACCAAACTGGATATAATC-3, antisense: 5-GGTCCTTAGCCACTCCTTCTGTG-3) and β-actin (sense: 5′-TGGAATCCTGTGGCATCCATGAAAC-3′, antisense: 5′-TAAAACGCAGCTCAGTAACAGTCCG-3′) were added thereto, and then each gene was amplified using Taq polymerase. At this time, a gene amplification condition is: 30 cycles of 30 sec. at 95° C., 1 min. at 55° C. and 1 min. at 72° C., and then one cycle of 5 min. at 72° C. The amplified cDNA was electrophoresized in a 1% agarose gel, and observed using a UV detector.

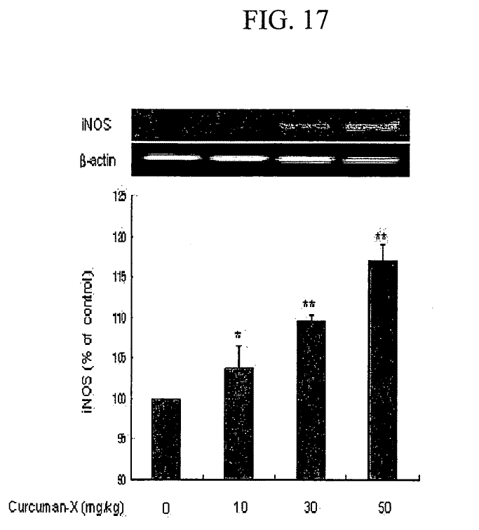

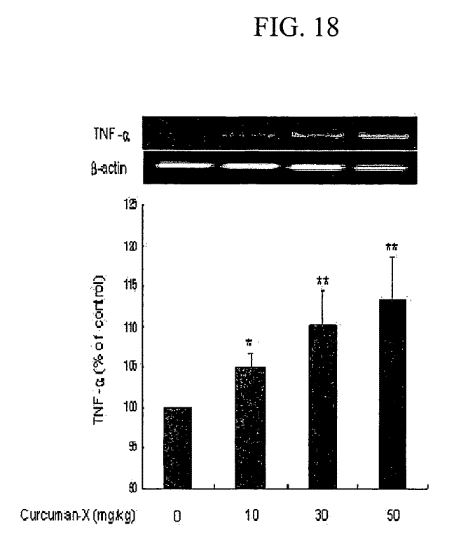

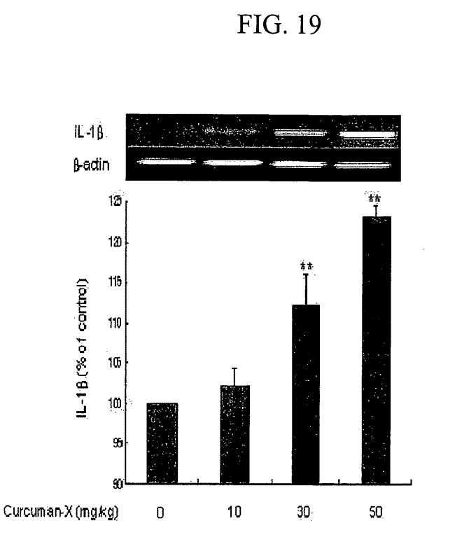

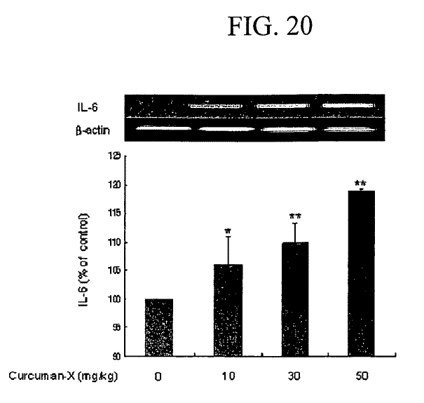

As a result, it was revealed that mRNAs of iNOS, TNF-α, IL-1β and IL-6 are clearly increased by the polysaccharides, as shown in FIG. 17, FIG. 18, FIG. 19 and FIG. 20. The results indicate that the immunostimulating effects of Curcuman-X as described in the Experimental examples are caused by the control of the mRNA expressions.

### Examples 2 to 13

### Isolation of Polysaccharides from Curcuma xanthorrhiza According to Extraction Condition

Polysaccharides were extracted, isolated and purified from Curcuma xanthorrhiza in the same manner as in Example 1, using hot water, 0.01˜5 N NaOH and 0.01˜5 N HCl as the extraction solvent of the polysaccharides. Subsequently, the NO production and the phagocytotic ability of the macrophage were measured in the same manner as in Experimental example 3 and Experimental example 5, except that the polysaccharides were used at a density of 10 ug/ml, and the results are listed in the following Table 4, and also represented by percent (%) based on results of the untreated group. As listed in Table 4, an extraction yield was 2.1 to 8.5% according to the extraction conditions, and the immunostimulating polysaccharides having a number average molecular weight of 11,000 to 82,000 were extracted and showed the NO production in all the extraction conditions.

TABLE 4

| | | | Number- | | |
|---|---|---|---|---|---|
| | | | Average | NO | |
| | Extraction | Yield | Molecular | Production | Phagocytosis |
| Example | Condition | (%) | Weight | (%) | (%) |
| 2 | Hot Water | 2.1 | 82,000 | 31.3 | 58.3 |
| 3 | 0.005N NaOH | 4.7 | 51,000 | 27.3 | 29.8 |
| 4 | 0.01N NaOH | 4.3 | 48,000 | 29.9 | 44.1 |
| 5 | 1N NaOH | 7.9 | 21,000 | 25.4 | 43.7 |
| 6 | 5N NaOH | 8.5 | 11,000 | 19.1 | 29.9 |
| 7 | 10N NaOH | 9.7 | 8,900 | 16.9 | 25.1 |
| 8 | 0.005N HCl | 3.8 | 57,000 | 24.5 | 49.3 |
| 9 | 0.01N HCl | 4.1 | 63,000 | 28.8 | 58.8 |
| 10 | 0.1N HCl | 4.9 | 47,000 | 24.5 | 57.4 |
| 11 | 1N HCl | 6.5 | 38,000 | 22.2 | 49.2 |
| 12 | 5N HCl | 7.7 | 29,000 | 14.0 | 31.1 |
| 13 | 10N HCl | 8.3 | 22,000 | 12.7 | 35.4 |

### Experimental Example 15

### In vivo Anti-Cancer of Curcuman-X

BDF1 mice (17-18 g, female) were divided into three groups, each group including 10 mice. Then, a B16F10 cancer cell was transplanted into peritoneal cavities of the mice in each of the groups to induce cancer, and, from the next day of the administration, the polysaccharides were diluted with saline and the mice were daily administered or ally once with the polysaccharides at densities of 10, 50 and 100 mg/kg. In order to assess toxicity in the experimental groups, the mice were weighed whenever they were treated with the samples, and the weight lose was not observed, which indicates that the toxicity is not present in all the experimental groups. As a result, a survival rate was represented by the number of the mice survived 60 days after the cancer cell transplantation, and also calculated as a ratio to a survival rate of the untreated mice.

As a result, the survival rate was increased in a dose-dependent manner by the treatment of Curcuman-X, as shown in the following Table 5, which indicates that Curcuman-X exhibits in vivo anticancer effect.

TABLE 5

| | | No. of Remaining Animal |
|---|---|---|
| Dose | Survival Rate (%) | (Total: 10) |
| 0 mg/kg | 0 | 0 |
| Curcuman-X 10 mg/kg | 20 | 2 |
| Curcuman-X 50 mg/kg | 50 | 5 |
| Curcuman-X 100 mg/kg | 70 | 7 |

### Experimental Example 16

### In vivo Anti-Tumor Effect of Curcuman-X on Cancer Cell

ICR mice (20-23 g, female) were divided into three groups, each group including 10 mice. Then, a cancer cell (sarcoma-180) was diluted with saline to obtain a cell solution (1×10⁶ cells), and 200 ul of the cell solution (1×10⁶ cells) was injected subcutaneously, and then an anticancer effect was measured using a solid cancer model. 24 days after the injection, the mice were daily administered orally once with the Curcuman-X at densities of 10, 50 and 100 mg/kg. A solid cancer developed 20 days after the sample injection was extracted to weigh the solid cancer. A suppression rate of solid cancer was calculated from the weight of the extracted solid cancer, and the results were listed in the following Table 6.

TABLE 6

| | Weight of Solid | Suppression of Solid |
|---|---|---|
| Dose | Cancer (mg) | Cancer (%) |
| 0 mg/kg | 449 ± 98 | 0 |
| Curcuman-X 10 mg/kg | 301 ± 36 | 30 |
| Curcuman-X 50 mg/kg | 276 ± 90 | 38.6 |
| Curcuman-X 100 mg/kg | 199 ± 81 | 62.1 |

As seen in Table 6, it was revealed that a weight of the solid cancer is 449±98 mg in the untreated group, and weights of the solid cancers are 276±90 mg and 199±81 mg in the experimental groups treated with 50 and 100 mg of Curcuman-X, respectively, and therefore the experimental groups exhibit a high suppression ratio of the solid cancer as much as 38.6 and 62.1%, respectively, compared to that of the control.

### INDUSTRIAL APPLICABILITY

The present invention provides the method for manufacturing effective polysaccharides from Curcuma xanthorrhiza, the polysaccharides obtained according to the manufacturing method, and the pharmaceutical composition comprising the polysaccharides as effective component. The polysaccharides and the pharmaceutical composition according to the present invention are useful in immunostimulating effect and anticancer effect.

1: A method for manufacturing polysaccharides isolated from Curcuma xanthorrhiza, comprising:
(S1) preparing a powder of Curcuma xanthorrhiza Roxb.;
(S2) extracting the powder with an organic solvent, and then filtering or centrifuging to obtain a residue;
(S3) extracting the residue to prepare a solution containing polysaccharides;
(S4) removing starch by adding starch-hydrolyzing enzyme to the polysaccharides-containing solution;
(S5) precipitating the polysaccharides after the step (S4); and
(S6) purifying the polysaccharides after the step (S5).

2: The method for manufacturing polysaccharides isolated from Curcuma xanthorrhiza according to claim 1,
wherein the extracting of the step (S3) is performed by using a hot water, an acid solution or an alkaline solution.

3: The method for manufacturing polysaccharides isolated from Curcuma xanthorrhiza according to claim 2,
wherein the alkaline solution is 0.005 to 10N NaOH solution.

4: The method for manufacturing polysaccharides isolated from Curcuma xanthorrhiza according to claim 2,
wherein the acid solution is 0.005 to 10N HCl solution.

5: The method for manufacturing polysaccharides isolated from Curcuma xanthorrhiza according to claim 1,
wherein the step (S5) is performed by adding lower alcohol after the step (S4).

6: The method for manufacturing polysaccharides isolated from Curcuma xanthorrhiza according to claim 1,
wherein the step (S6) is performed by removing low molecular weight components using dialysis or ultra-filtration.

7: Polysaccharides isolated from Curcuma xanthorrhiza, obtained by the manufacturing method as defined in claim 1.

8: A pharmaceutical composition comprising the polysaccharides as defined in claim 7 as effective component.

9: An anticancer supplementary composition comprising the polysaccharides as defined in claim 7 as effective component.

10: An immunostimulating composition comprising the polysaccharides as defined in claim 7 as effective component.


SEQUENCE LISTING (8 sequences)

SEQ ID NO: 1 - DNA, 20 bp, Artificial Sequence
  sense primer for iNOS
caaccagtattatggctcct

SEQ ID NO: 2 - DNA, 20 bp, Artificial Sequence
  antisense primer for iNOS
gtgacagcccggtctttcca

SEQ ID NO: 3 - DNA, 20 bp, Artificial Sequence
  sense primer for TNF-alpha
cctgtagcccacgtcgtagc

SEQ ID NO: 4 - DNA, 20 bp, Artificial Sequence
  antisense primer for TNF-alpha
ttgacctcagcgctgagttg

SEQ ID NO: 5 - DNA, 19 bp, Artificial Sequence
  sense primer for COX-2
ccgtggtaatgtatgagca

SEQ ID NO: 6 - DNA, 19 bp, Artificial Sequence
  antisense primer for COX-2
ctcgcttctgatatgtctt

SEQ ID NO: 7 - DNA, 25 bp, Artificial Sequence
  sense primer for beta-actin
tggaatcctgtggcatccatgaaac

SEQ ID NO: 8 - DNA, 25 bp, Artificial Sequence
  antisense primer for beta-actin
taaaacgcagctcagtaacagtccg